(19) United States
(12) Patent Application Publication
(10) Pub. No.: US 2013/0231690 A1
(43) Pub. Date: Sep. 5, 2013

(54) Plaque removal tool

(71) Applicant: John Lucian Costley, JR., Palmetto, FL (US)

(72) Inventor: John Lucian Costley, JR., Palmetto, FL (US)

(21) Appl. No.: 13/135,454

(22) Filed: Jul. 5, 2011

(51) Int. Cl. A61B 17/3207

(52) U.S. Cl. CPC A61B 17/320725
USPC 606/159

(57) ABSTRACT

The Plaque Removal Tool (PRT-01) is a device for the removal of plaque in the human body. The (PRT-01) has an inner and outer annulus body. The outer annulus body forms a guide for the purpose of alignment of the tool in the arteries. In the retracted mode the PRT-01 cuts away at an artery partially blocked with plaque using the cutting face at the front of the tool. For fully blocked arteries the tool cuts away at the blocked artery until the time that it can no longer advance. At that time, the tool is extended and the carbide cutting arms are exposed to the plaque on the arterial wall. This way the carbide cutting arms remove enough plaque to allow the outer annulus body to advance. The inner tube gets applied torque from the drive motor, this motion is lubricated through the inner tube.

The Plaque Removal Tool is a tool to assist doctors to be able to remove arterial plaque from the human body. The PRT consists of an outer annulus that allows the engagement of the tool in separate and complimentary fashions to remove the plaque from either a partially blocked artery or a completely blocked artery. The outer annulus body contains an inner annulus that has several internal parts. This inner annulus holds the carbide cutting arms(6). These arms are held in by a “Camelback” style of hump where they cannot extend without the inner annulus being extended. These arms are activated when the tool is extended and fit through notches cut in the outer surface of the outer annulus. The carbide arms' cutting faces are pulled up through the outer annulus by a pair of springs attached to each carbide arm. Each carbide arm is held in place at the rear of the carbide cutting arms by a hinge attached to the center disc. The disc at the front of the tool, the cutting face disc is faced with carbides to allow the PRT tool to cut away at a partially blocked artery. In the case of a completely blocked artery the PRT advances until it cannot advance, at that time the inner annulus of the PRT tool is extended by a feed of the fluid drive inlet tube that is hollow and reinforced to facilitate the torque motion and the input of fluid to lubricate the cutting face thus allowing the detritus to flow to a dialysis machine through the return fluid tube that is held in place by the locking ring so that the bits of removed plaque are kept from circulation in the body of the patient.

There are three discs, the front disc that has a carbide face, the center disc that holds the Hinges for the carbide cutting arms and the rear disc that forms the rear of the body of the PRT. These discs are held in true alignment by three threaded rods that keep the alignment of the PRT tool true and at the proper axis for use.

The fluid inlet tube is driven by an electric motor (by others), it is inside of the fluid return tube where isolation and fluid balance of the body can be maintained and the detritus of plaque can be separated by a dialysis machine thereby reducing the inherent problems of free floating plaque.

### THE DRAWINGS

FIG. 1 is an isometric drawing of the complete tool. 1. Is the Outer Annulus. 2>Is the inner Annulus. 3>is the Front Carbide disc. 4>Is the Center Disc. 5>Is the Rear Disc. 6>Is the Carbide Arm (there are three). 7>are the Springs (there are six) 8>Is the Hinge (there are three) 9>These are the threaded rods (there are three). 10>Is the locking ring for the Return Fluid Tube. 11>Is the drive Fluid inlet. 12>Is the return Fluid Tube. What is not shown is the entire length of the fluid drive and return fluid drive hoses.

FIG. 2 is an isometric drawing of The Drive motor and its arrangement.

FIG. 3 is an isometric drawing of the outer annulus (1).

FIG. 4 is an isometric drawing of the inner annulus

FIG. 5 is a frontal drawing view of the Front disc with a carbide face (3).

FIG. 6 is a drawing of the (4) center disc.

FIG. 7 is a drawing of (5) the rear disc.

FIG. 8 is a drawing of the view The Front View of (6) the carbide arm (three required).

FIG. 9 is a top view of the carbide Arm (6) and the springs (7) required to activate the Carbide Arm.

FIG. 10 is a side view of The Carbide arm (6) and the spring (7)

FIG. 11 is a drawing of (7) the springs (6 required)

FIG. 12 is a front view of the hinge (8)

FIG. 13 is a view of the treaded rod (9) three required

FIG. 14 is a drawing of (10) the locking ring (one required).

FIG. 15 is a drawing of the drive assembly consisting of (11) the drive fluid inlet hose, (12) the return fluid tube and (13) the drive motor and (14) two “O” rings to isolate the fluid from the drive motor.

FIG. 16 is a drawing of the side view of the Plaque Removal Tool (in the retracted position.) 1>Is the Outer Annulus. 2>Is the inner Annulus. 3>is the Front Carbide disc. 4>Is the Center Disc. 5>Is the Rear Disc. 6>Is the Carbide Arm (there are three). 7>are the Springs (there are six) 8>Is the Hinge (there are three) 9>These are the threaded rods (there are three). 10>Is the locking ring for the Return Fluid Tube. 11>Is the drive Fluid inlet. 12>Is the return Fluid Tube. What is not shown is the entire length of the fluid drive and return fluid drive hoses.

FIG. 17 is a drawing of the side view of the Plaque Removal tube in the extended position. 1>Is the Outer Annulus. 2>Is the inner Annulus. 3>is the Front Carbide disc. 4>Is the Center Disc. 5>Is the Rear Disc. 6>Is the Carbide Arm (there are three). 7>are the Springs (there are six) 8>Is the Hinge (there are three) 9>These are the threaded rods (there are three). 10>Is the locking ring for the Return Fluid Tube. 11>Is the drive Fluid inlet. 12>Is the return Fluid Tube. What is not shown is the entire length of the fluid drive and return fluid drive hoses.

FIG. 18 is a drawing of the top view of the Plaque Removal tube in the retracted position. 1>Is the Outer Annulus. 2>Is the inner Annulus. 3>is the Front Carbide disc. 4>Is the Center Disc. 5>Is the Rear Disc. 6>Is the Carbide Arm (there are three). 7>are the Springs (there are six) 8>Is the Hinge (there are three) 9>These are the threaded rods (there are three). 10>Is the locking ring for the Return Fluid Tube. 11>Is the drive Fluid inlet. 12>Is the return Fluid Tube. What is not shown is the entire length of the fluid drive and return fluid drive hoses.

FIG. 19 is a drawing of the side view of the Plaque Removal tube in the extended position. 1>Is the Outer Annulus. 2>Is the inner Annulus. 3>is the Front Carbide disc. 4>Is the Center Disc. 5>Is the Rear Disc. 6>Is the Carbide Arm (there are three). 7>are the Springs (there are six) 8>Is the Hinge (there are three) 9>These are the threaded rods (there are three). 10>Is the locking ring for the Return Fluid Tube. 11>Is the drive Fluid inlet. 12>Is the return Fluid Tube. What is not shown is the entire length of the fluid drive and return fluid drive hoses.

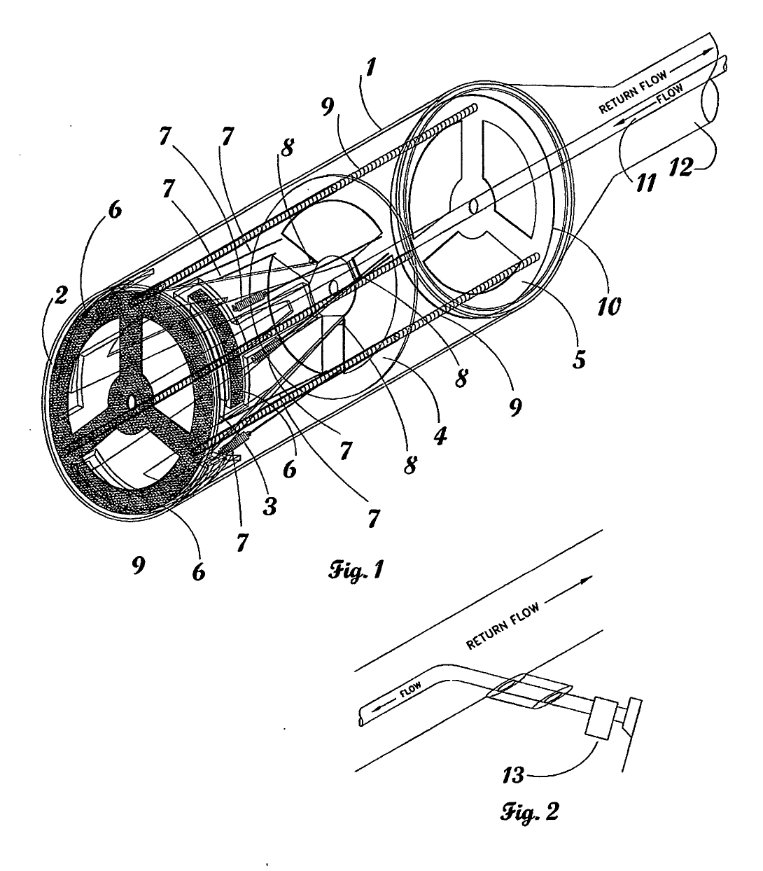

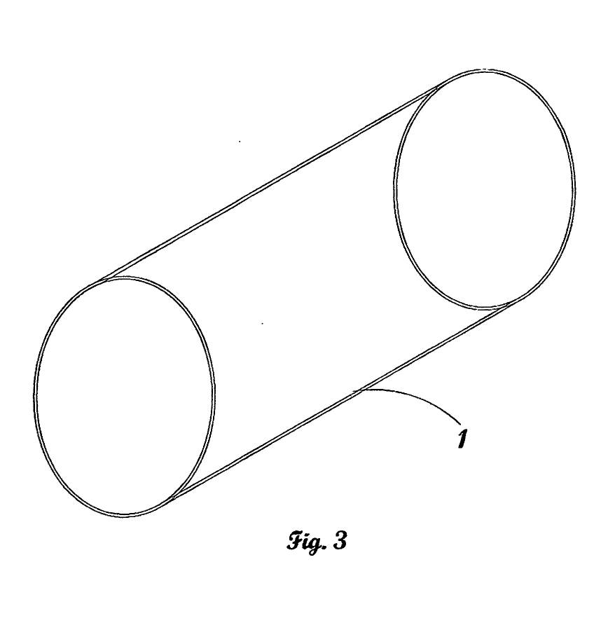

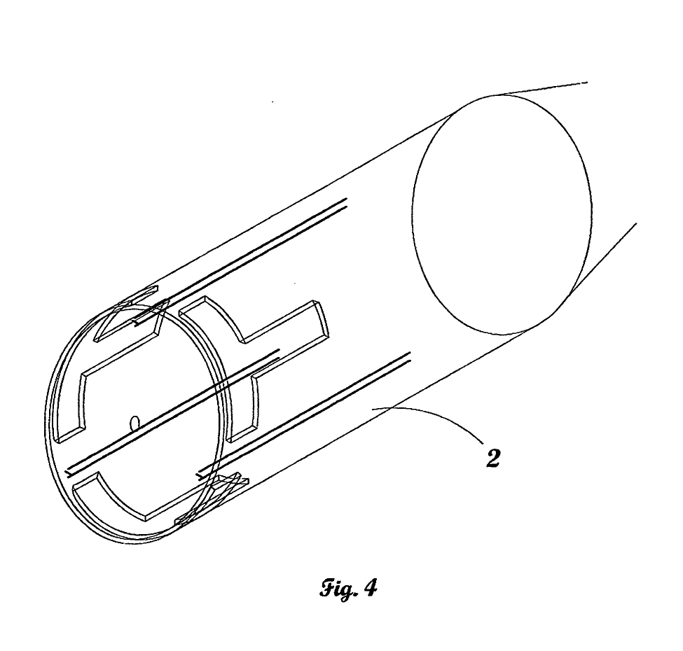

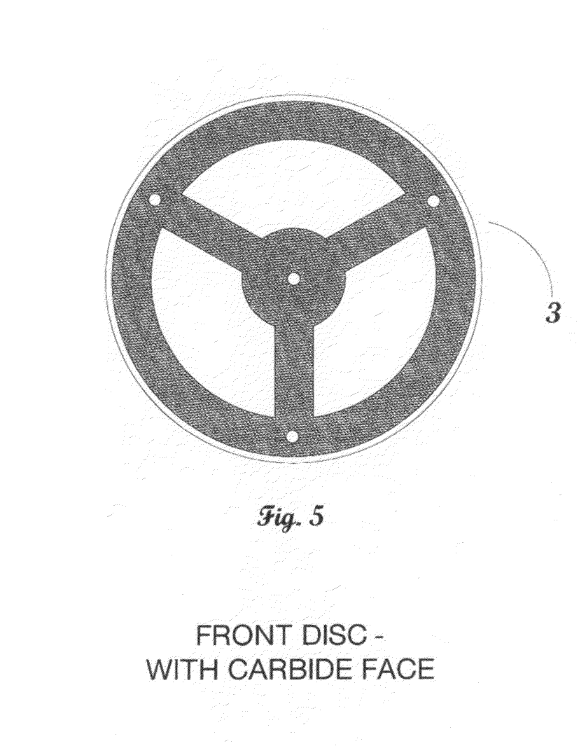

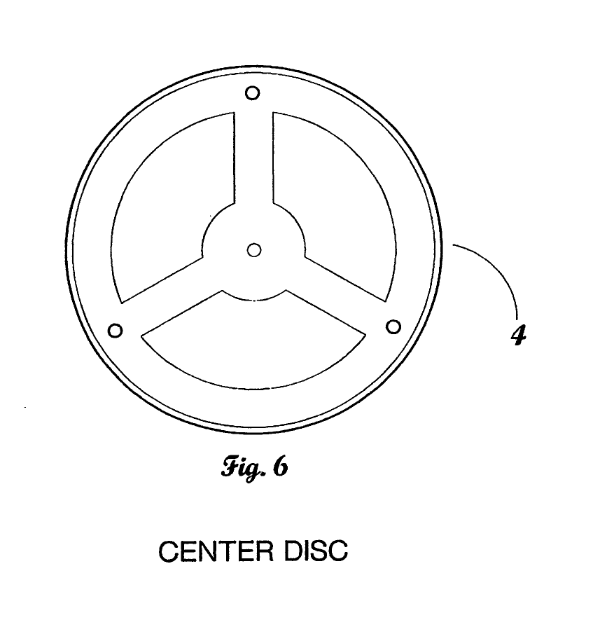

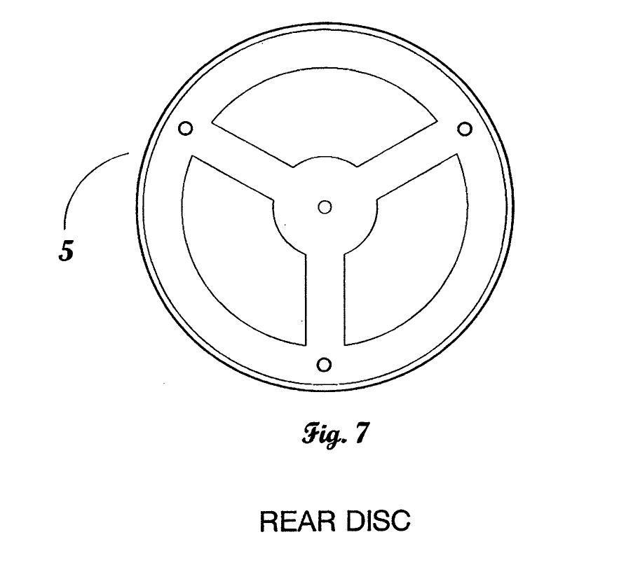

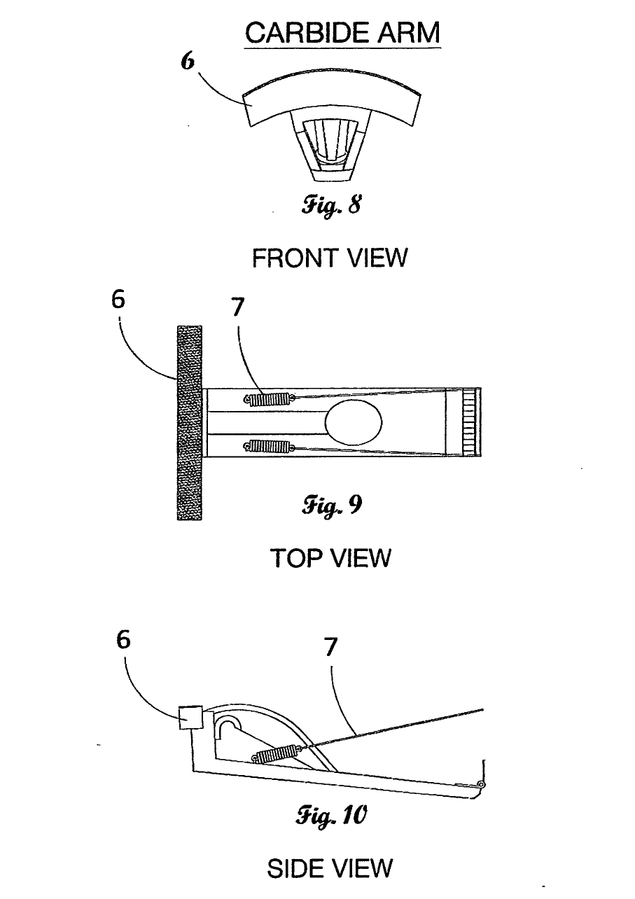

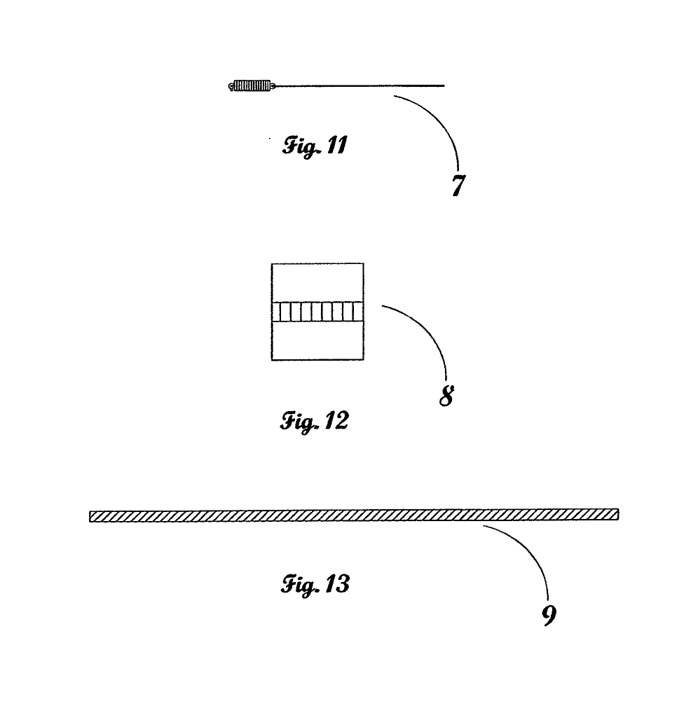

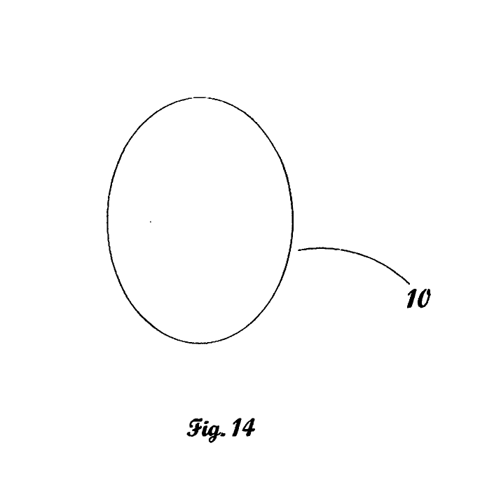

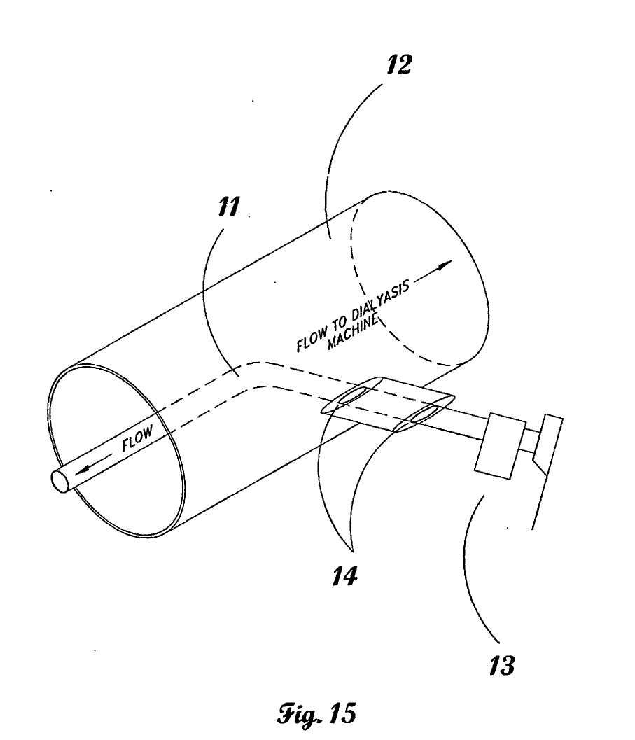

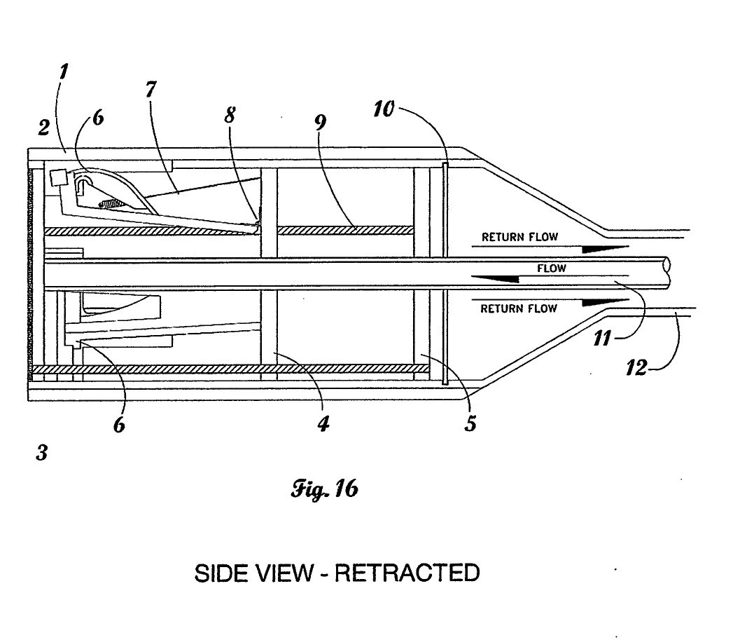

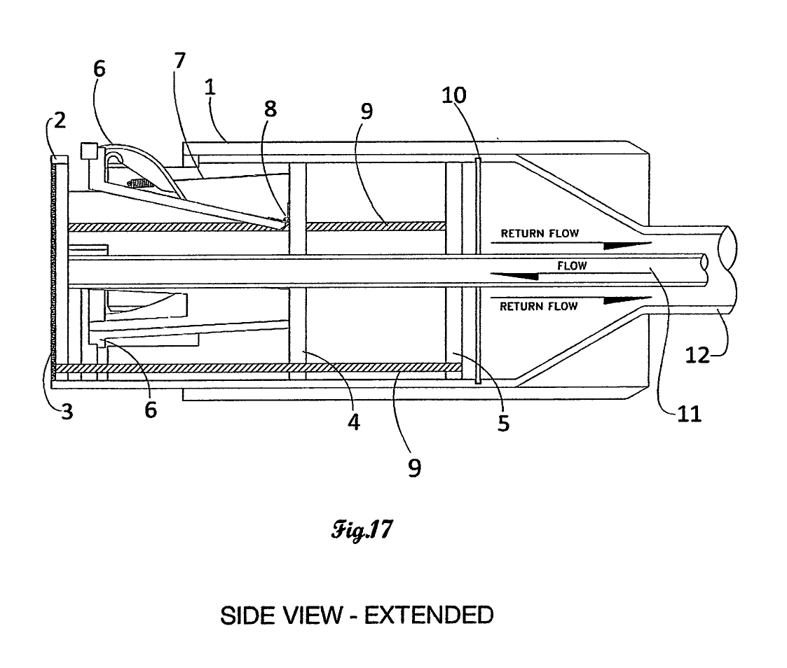

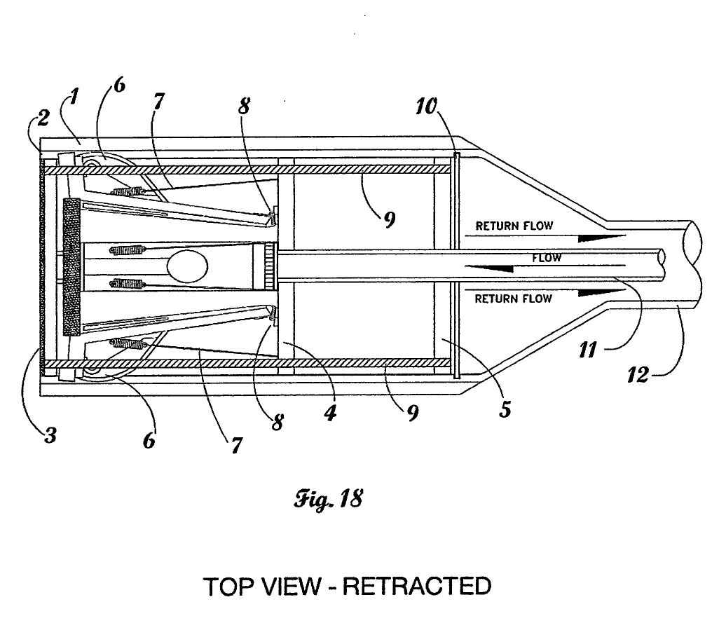

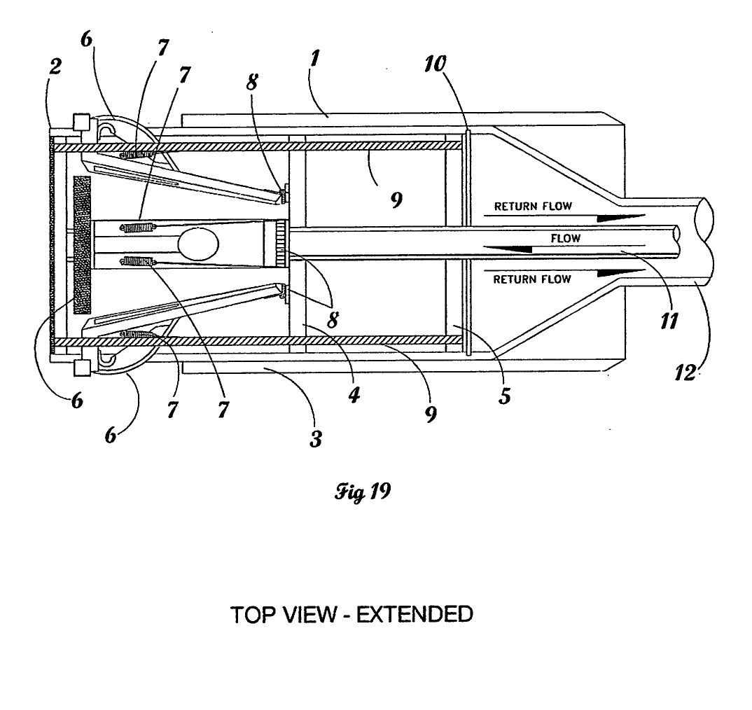

1. A tool for the removal of arterial plaque, be it partial or a complete blockage, comprised of an outer annulus with a return tube locked into the inner surface of the annulus, an extended position that side cuts with extending and retracting carbide arms to allow the forward passage of the outer annulus, a retracted position that allows only the cutting face to the blockage for partially blocked arteries, a drive shaft that also is the passageway for the imput of liquid to the cutting face and a return tube for the removal of cut away plaque and detritus thereby minimizing risk to the patient, a motor to turn the imput shaft and control the extension and retraction of the inner annulus of the two.

2. The tool torque of the return fluid tube is countered by the wye and acts as a concentric centering device for the tool while it is removing partial or total plaque blockages from an arterial wall.

3. The outer annulus is further provided with a means to extend the tool that allows the carbide arms to be exposed thereby providing a way to clear away plaque to allow the advancement of the outer annulus. This also allows fluid to be sent in via the drive fluid inlet tube and provide a way that a stable fluid balance is maintained in the body of the patient.

4. The outer annulus is provided with a tubular body as is the inner tool body with tolerance to rotate the tool and carbide arms thereby providing a concentric centering of both tool bodies while working either in the retraced or extended positions. This is further enhanced by the control of the pressure dynamics of the blood pressure in the body by the addition of fluid while removing blood and plaque detritus.

5. The inner annulus is further provided with movable carbide arms that can cut away at plaque while the face of the tool is cutting away at plaque thereby providing a space clearance for the outer annulus. These arms extend and retract by means of the movement forward or backward of the fluid inlet drive tube that provides torque and fluid and movement of the carbide arms. The inner annulus also has “ T ” shaped cutouts to allow space for the movement of the carbide arms.
